(19) United States
(12) Patent Application Publication
(10) Pub. No.: US 2011/0066028 A1
(43) Pub. Date: Mar. 17, 2011

(54) SYSTEMS AND METHODS FOR REMOTE MONITORING OF IMPLANTABLE MEDICAL DEVICE LEAD TEMPERATURES DURING AN MRI PROCEDURE

(75) Inventors: Xiaoyi Min, Thousand Oaks, CA (US); Gene A. Bornzin, Simi Valley, CA (US)

(73) Assignee: PACESETTER, INC., Sylmar, CA (US)

(21) Appl. No.: 12/558,366

(22) Filed: Sep. 11, 2009

(51) Int. Cl. A61B 5/01; A61N 1/08; A61B 5/055

(52) U.S. Cl. 600/412; 607/119

(57) ABSTRACT

Systems and methods are provided for detecting and responding to excessive heating of implantable medical device leads, such as leads used with pacemakers or implantable cardioverter-defibrillators (ICDs), during a magnetic resonance imaging (MRI) procedure. In one example, a critical temperature is determined for the lead that is representative, e.g., of the temperature at which tissue damage might occur or pacing/sensing might be significantly impaired. A temperature threshold is then set based on the critical temperature by subtracting a predetermined safety margin. Lead temperatures are then sensed during the MRI procedure. The lead temperatures are compared against the threshold and suitable warnings are transmitted to an external monitoring system if lead temperatures exceed their thresholds so that the attending personnel can take corrective action. The implantable device may also be programmed to take corrective action, such as automatically changing pacing modes, adjusting pulse magnitudes or sensitivity values, etc.

### FIELD OF THE INVENTION

The invention generally relates to implantable medical devices, such as pacemakers or implantable cardioverter/defibrillators (ICDs), and to external diagnostics systems for use therewith and, in particular, to techniques for tracking lead temperatures within a patient during a magnetic resonance imaging (MRI) procedure.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed radio-frequency (RF) magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

However, MRI procedures are problematic for patients with implantable medical devices such as pacemakers and ICDs. One of the significant problems or risks is that the strong RF fields of the MRI can induce currents through the lead system of the implantable device into the tissues resulting in Joule heating in the cardiac tissues around the electrodes of leads, potentially damaging adjacent tissues. Indeed, in worst-case scenarios, the temperature at the tip of an implanted lead has been found to increase as much as 70 degrees Celsius (C.) during an MRI tested in a gel phantom in a non-clinical configuration. Although such a dramatic increase is probably unlikely within a clinical system wherein leads are properly implanted, even a temperature increase of only about 6°-13° C. might cause myocardial tissue damage.

Furthermore, any significant heating of the electrodes of pacemaker and ICD leads, particular tip electrodes, can affect pacing and sensing parameters associated with the tissue near the electrode, thus potentially preventing pacing pulses from being properly captured within the heart of the patient and/or preventing intrinsic electrical events from being properly sensed by the device. The latter may potentially result, depending upon the circumstances, in therapy being improperly delivered or improperly withheld. Therefore techniques to reduce heating of within medical device leads, especially leads of pacemakers and ICDs, are particularly desirable.

Techniques have been developed for detecting the heating of tip electrodes during an MRI procedure. See, for example, U.S. Patent Application 2006/0025820, of Phillips et al., entitled “Integrated System and Method for MRI-safe Implantable Devices.” The implanted device described therein is equipped to measure tip temperatures and to communicate temperature information or an indication of a potential heating condition to an external system. For example, a logic signal may be provided to the MRI system indicating that heating has exceeded a threshold so that an MRI procedure can then be terminated. Alternatively, raw electrical signals and/or temperature information can be provided to the MRI system so that the MRI sequence can be terminated or adjusted to reduce potential heating. Still further, the system can communicate the temperature information or an indication of a potential heating condition to an external device programmer. Insofar as the thresholds are concerned, the document describes that a baseline temperature for the lead is determined prior to an MRI diagnostic procedure and the threshold is set relative to the baseline, such as corresponding to a predetermined number of degrees greater than the baseline temperature (e.g., approximately two degrees Fahrenheit greater.) That is, the threshold is set so as to detect some relatively modest increase in tip temperature. A possible concern with this type of procedure is that the MRI procedure may then be automatically terminated or manually adjusted even though the tip temperatures are well below the critical temperature at which tissue damage might occur or at which sensing/pacing might be impaired.

Accordingly, it would be desirable to provide improved techniques for measuring and tracking tip temperatures during MRIs and also for providing enhanced diagnostic information regarding tip temperatures and aspects of the present invention are directed to that end. For example, it would be desirable to provide improved techniques for setting tip temperature thresholds to optimal values so as to permit suitable warnings to be generated before a damaging temperature is reached so that corrective action can then be taken, but which do not trigger unnecessary warnings or actions in response to modest increases in tip temperatures.

### SUMMARY OF THE INVENTION

In accordance with an exemplary embodiment of the invention, a method is provided for use by an implantable medical device for implant within a patient wherein the device uses at least one electrical lead implanted within patient tissue. The method is directed to controlling at least one device function based on lead temperatures during an MRI. In one example, a critical temperature is determined for the lead representative, e.g., of the temperature at which tissue damage might occur or pacing/sensing might be significantly impaired. A temperature threshold is then set based on the critical temperature to a value below the critical temperature. For example, a predetermined safety margin may be subtracted from the critical temperature so as to derive the threshold temperature. Lead temperature values are then sensed during an MRI procedure or other magnetic imaging procedure. The lead temperature values are compared against the threshold and devices functions are then controlled based on whether the lead temperature exceeds the threshold, such that device functions can be controlled in response to lead temperatures before the temperatures approach the critical temperature.

In particular, the implantable device is preferably controlled to transmit warning signals and lead temperature values to an external monitoring system for display thereon so that the attending personnel can take corrective action. Depending upon the actual temperature values, the personnel can, for example, selectively suspend the MRI system or otherwise control its operation to address the rising lead temperatures. Alternatively, the personnel might adjust the operations of the implanted device via long range telemetry to address the rising tip temperatures, such as by changing pacing modes or the like. By using a threshold set relative to the critical temperature (by, e.g., subtracting a predetermined safety margin therefrom), warnings are not unnecessarily triggered in response to relatively modest temperature increases within the lead. Rather, warnings are only generated if temperatures begin to approach the critical temperature. A suitable safety margin may be, e.g., in the range of 3 to 4° C.

In one particular example, the critical temperature for the lead is determined or estimated in advance and stored within device memory for subsequent retrieval. Alternatively, the device is equipped to calculate the critical temperature for a particular lead based on temperature models or the like stored within the device. Typically, two or more leads are provided for use with the device and so a different critical temperature may be specified for each lead. Different safety margins may also be specified for use with different leads. Preferably, temperature sensors are mounted near the tip electrode of the leads so as to measure tip temperatures, as the tip is usually experiences the most heating during an MRI. The critical temperature is thus determined relative to tip temperatures. In some implementations, the temperature sensor is only activated in response to detection of an MRI field. In other implementations, it may remain active at all times to track cardiac temperature. Fiber optic-based temperature sensors are particularly appropriate as such devices can typically be configured to provide reliable temperature measurements even in the presence of strong magnetic fields.

Alternatively, rather than implementing the temperature monitoring procedures within the implantable device, some or all of the analysis procedures may be implemented within an external system, such as within the external monitor or within a device programmer. That is, in one embodiment of the invention, a method is provided for use with an external monitoring system used in conjunction with an implantable medical device having at least one lead for implant within a patient. The method includes: determining a critical temperature of the lead; setting a temperature threshold based on the critical temperature to a value below the critical temperature; receiving lead temperature values from the implanted device via telemetry during an MRI or other magnetic imaging procedure; comparing the lead temperature values against the threshold; and controlling system functions based on whether the lead temperature values exceed the threshold, such that system functions thereby can be controlled in response to lead temperatures approaching the critical temperature. In particular, the external monitor is preferably controlled to display warning signals and lead temperature values so that the attending personnel can take corrective action. As with the device-based embodiments summarized above, by using a threshold set relative to the critical temperature (rather than, e.g., relative to a pre-MRI baseline), warnings are not unnecessarily triggered in response to relatively modest temperature increases within the lead of the implanted device. Rather, warnings are only generated if temperatures begin to approach the critical temperature. In some implementations, the warning signals and temperature values may be forwarded to a remote monitoring terminal positioned at a location remote from an MRI system performing the MRI.

These techniques are perhaps most advantageously implemented for use with MRI systems but principles of the invention may be exploited for use with other systems providing strong magnetic fields as well.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of an MRI system along with a patient with a pacer/ICD implanted therein that is capable of generating warnings of high tip temperatures during an MRI procedure for transmission to an external monitoring system;

FIG. 2 is a flow diagram providing an overview of lead temperature processing techniques performed by the pacer/ICD and the external monitoring system of FIG. 1;

FIG. 3 is a flow diagram providing a more detailed illustration of exemplary tip temperature processing techniques performed by the pacer/ICD in accordance with the general method of FIG. 2;

FIG. 4 is a flow diagram illustrating exemplary temperature processing techniques performed by the external monitor of FIG. 1 in accordance with an alternative embodiment wherein the external monitor tracks tip temperatures based on signals received from the pacer/ICD;

FIG. 5 is a simplified, partly cutaway view, illustrating the pacer/ICD of FIG. 1 along with a more complete set of leads implanted in the heart of the patient;

FIG. 6 is a functional block diagram of the pacer/ICD of FIG. 5, illustrating basic circuit elements that provide cardioversion, defibrillation and/or pacing stimulation in four chambers of the heart and particularly illustrating components for tracking and responding to increases in lead temperatures;

FIG. 7 is a functional block diagram illustrating components of an external monitoring system for use in receiving, analyzing and displaying temperature signals received from the pacer/ICD of FIG. 6 during an MRI procedure; and

FIG. 8 is a stylized representation of a set of exemplary implantable medical device leads showing temperature sensors at various locations along a lead.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Lead Temperature-Responsive Systems and Procedures

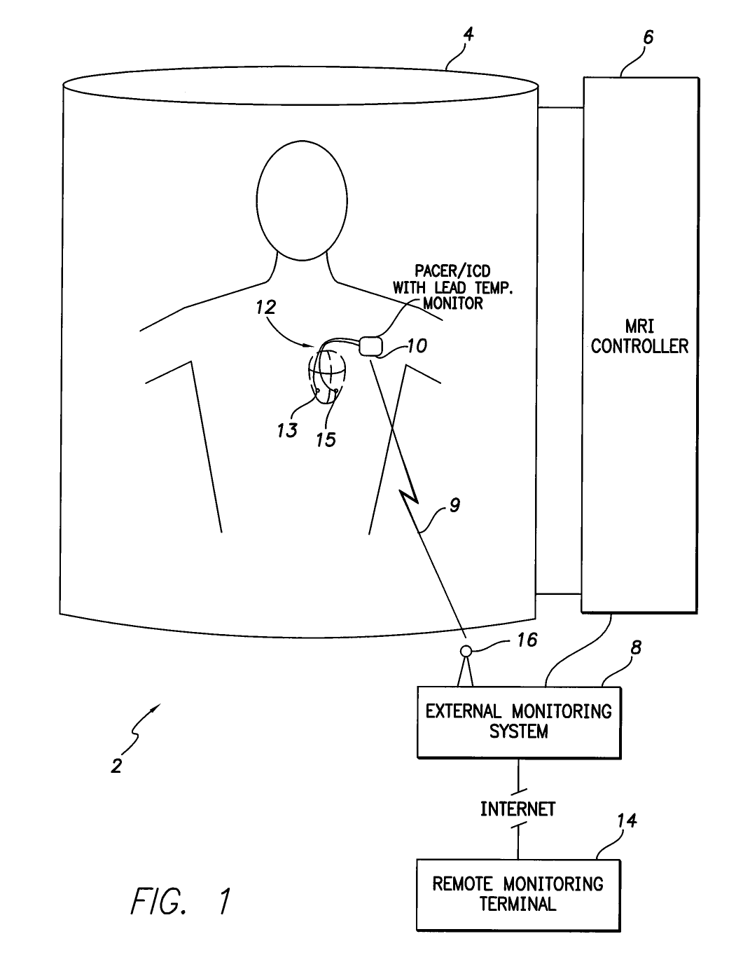

FIG. 1 illustrates an overall MRI system 2 having an MRI machine 4 operative to generate MRI fields during an MRI procedure for examining a patient. The MRI machine operates under the control of an MRI controller 6, which controls the strength and orientation of the fields generated by the MRI machine and derives images of portions of the patient therefrom, in accordance with otherwise conventional techniques. MRI machines and imaging techniques are well known and will not be described in detail herein. See, for example, U.S. Pat. No. 5,063,348 to Kuhara et al., entitled “Magnetic Resonance Imaging System” and U.S. Pat. No. 4,746,864 to Satoh, entitled “Magnetic Resonance Imaging System.” An external monitoring system 8 is also provided that communicates using antenna 16 via long range RF telemetry along communication link 9 with a pacer/ICD 10 implanted within the patient so as to receive transmissions of lead temperature warning signals and/or lead temperature values measured within the patient by the pacer/ICD during the MRI procedure, as well as other diagnostic data. Depending upon the nature of the warning, the external monitoring system may deactivate the MRI machine by sending appropriate control signals to the MRI controller. Alternatively, medical personnel operating the system may manually deactivate or adjust the MRI machine in response to such warnings by using an MRI control interface, not separately shown, or may take other corrective actions such as selectively reprogramming the operations of the pacer/CD.

Note that the external monitoring system may be positioned outside of the MRI room. If so, a telemetry unit of the monitoring system can be positioned inside the (shielded) MRI room to receive signals from the implantable device. The signals are then relayed to the external monitoring system via a shielded cable.

The pacer/ICD and the external monitoring system preferably employ filtering and transmission techniques described in U.S. patent application Ser. No. 11/938,088 of Min et al., filed Nov. 9, 2007, entitled “Systems and Methods for Remote Monitoring of Signals Sensed by an Implantable Medical Device during an MRI”, which is incorporated by reference herein. These filtering and transmission techniques allow for reliable transmission of signals between the pacer/ICD and the external monitor despite the presence of the MRI fields.

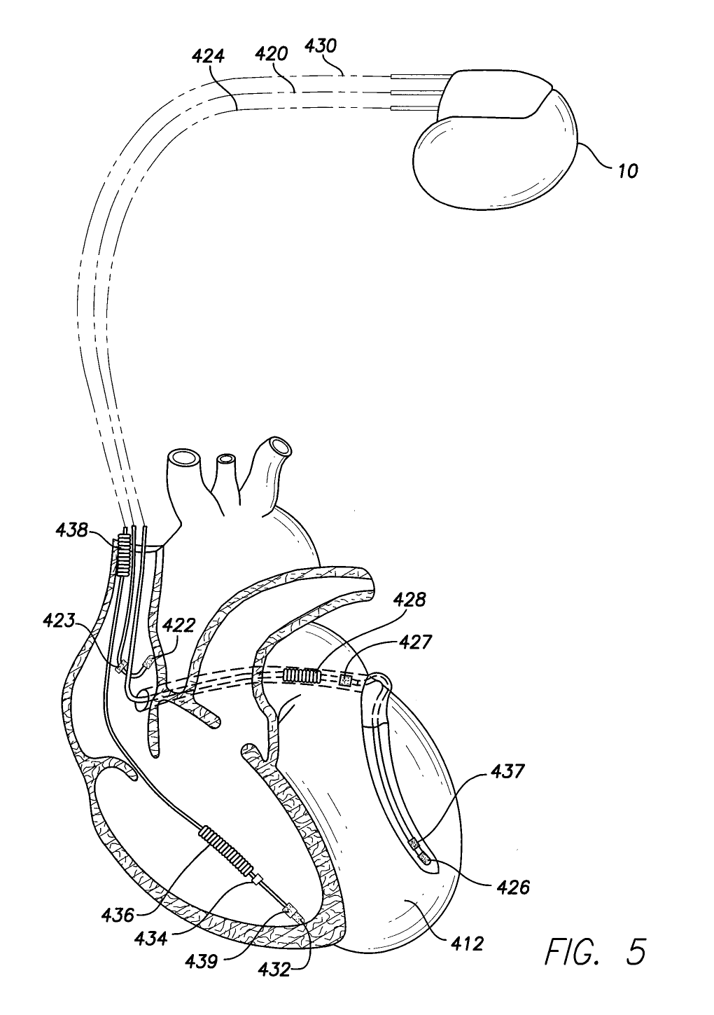

A lead system 12 is coupled to pacer/ICD 10 for sensing electrophysiological signals within the heart of the patient, such as A-IEGM and V-IEGM signals, and for delivering any needed pacing pulses, shock therapy or other electrical stimulation therapy. Temperature sensors 13 and 15 are provided near the tips of the leads for measuring tip temperatures during the MRI procedure so that the pacer/ICD can track tip temperature during the MRI and forward appropriate warning signals to the external monitor, if needed. Particular techniques for determining when to generate and transmit such warning signals are discussed below. In FIG. 1, only two leads are shown, each with a single temperature sensor. Additional leads and/or additional temperature sensors may be employed. A more complete lead system is illustrated in FIG. 5, described below. The lead system may also include various physiological sensors (not shown) for sensing hemodynamic signals or other signals within the patient, such as sensors operative to sense intracardiac pressure, blood oxygen saturation (i.e. blood SO₂), etc. In some cases, the sensors may be implanted elsewhere in the patient or may be mounted in or on the pacer/ICD itself. In any case, any of the various hemodynamic signals or other signals sensed using the sensors might potentially be transmitted to the external monitoring system during the MRI procedure for display thereon along with the aforementioned lead temperature-based signals. Where appropriate, temperature signals or other diagnostic information received or generated by the external monitoring system is forwarded via the Internet or other appropriate communications network to a remote monitoring terminal 14 for review thereon.

### Lead Temperature Tracking Procedures

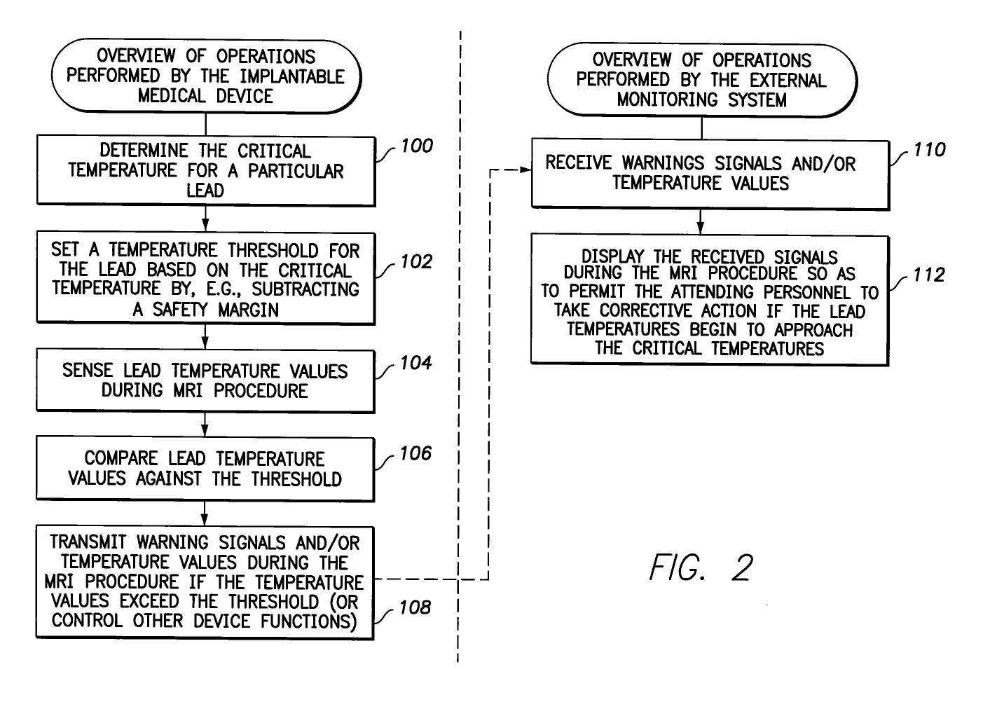

FIG. 2 broadly summarizes the operations performed by the pacer/ICD and the external monitoring system of FIG. 1 for tracking lead temperatures during an MRI and for responding thereto. Briefly, beginning at step 100, the pacer/ICD determines the critical temperature for a particular lead, i.e. the temperature at which tissue damage might occur or pacing/sensing might be significantly impaired. Typically, critical temperatures for each of the various leads (that are at risk of excessive tip heating) are determined or estimated in advance and stored in device memory so that the pacer/ICD need only retrieve the values from memory. However, techniques that are more sophisticated may be employed, as discussed below, wherein the pacer/ICD calculates the critical temperature. At step 102, the pacer/ICD sets a temperature threshold for the lead based on the critical temperature by, e.g., subtracting a predetermined safety margin from the critical temperature. Thus, if the critical temperature for the LV lead is, e.g., 44° C. (i.e. about 8° C. above normal body temperature), and the safety margin is, e.g., 3° C., then the threshold temperature for the LV lead is thereby set to 41° C.

At step 104, the pacer/ICD senses lead temperature values during the MRI procedure using the temperature sensor in the lead. Then, at step 106, the pacer/ICD compares the lead temperature values against the threshold. If two or more leads are equipped with temperature sensors, then temperature signals from the various sensors are separately compared against respective thresholds determined for those particular leads. In any case, at step 106, the pacer/ICD generates and transmits warning signals during the MRI procedure to the external monitor if any of the temperature values exceed their respective thresholds. For example, if the tip temperature of the LV lead has surpassed a threshold set to 41° C., the pacer/ICD generates and transmits a warning signal specifying that the LV lead is undergoing excessive heating. Additionally, or alternatively, the raw temperature values may be transmitted. Still further, device functions can be controlled in response to the excessive hearting. For example, the pacer/ICD can be switched to an asynchronous pacing mode (such as AOO, VOO or DOO) so that any impairment in the sensing of cardiac signals (such as P-waves and R-waves) due to the rising tip temperatures does not interfere with the determination of whether pacing pulses should be delivered. This is particularly appropriate for use within pacemaker-dependent patients. As another example, the device might increase pulse amplitudes so that any impairment in the capture of pacing pulses (such as A-pulses and V-pulses) due to the rising tip temperatures does not interfere with the delivery of pacing pulses. This is likewise particularly appropriate within pacemaker-dependent patients.

Turning now to the operations performed by the external monitoring system, at step 110, the monitor receives warnings signals and/or temperature values from the pacer/ICD and, at step 112, displays the received signals during the MRI procedure so as to permit the attending personnel to take corrective action. As already noted the personnel might, depending upon the circumstances, suspend the MRI procedure or adjust the MRI machine in an effort to reduce tip heating. In any case, by providing an overall system that generates and displays temperature warnings based on thresholds set relative to the critical temperature for the lead (rather than set to some pre-MRI baseline temperature or the like), warnings are only generated if the temperature of the lead actually begins to approach the temperate at which some damage or impairment might occur. Hence, warnings are not generated in response to only a modest increase in tip temperatures. This allows MRI procedures to be completed in circumstances where the procedure might otherwise be terminated. This also allows the pacer/ICD to continue to operate in normal pacing/sensing modes despite elevated tip temperatures unless and until such temperatures approach a point above which normal pacing/sensing might be impaired. Other advantages may be achieved as well. Depending upon the implementation, the main steps of tracking lead temperatures and generating warning signals (or controlling device operations) may be performed either by the pacer/ICD (as shown in FIG. 2) or by the external monitoring system based on raw temperature signals sent by the pacer/ICD.

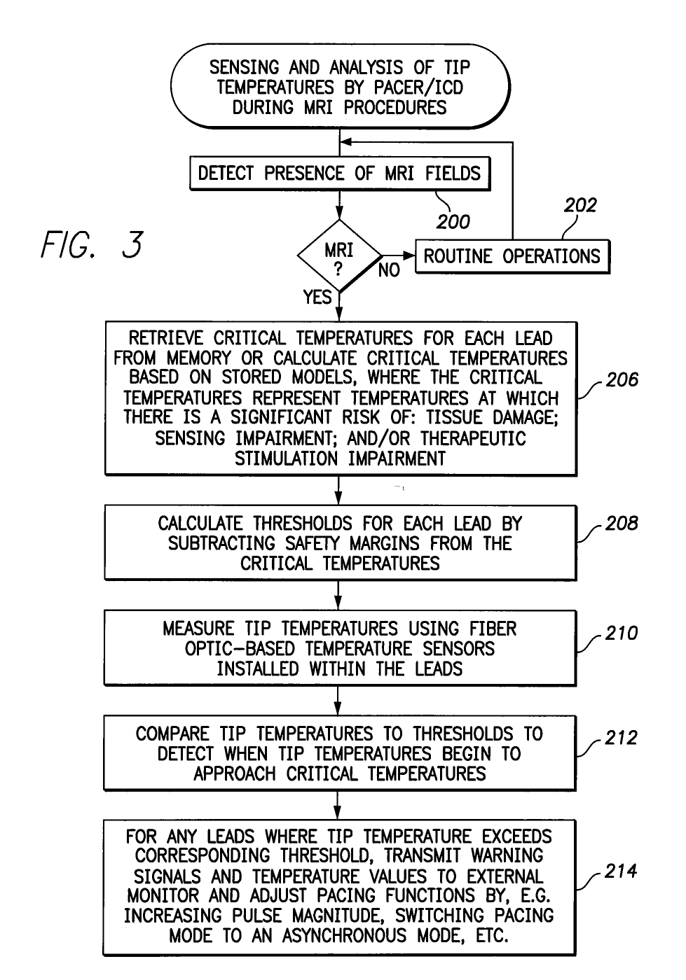

FIG. 3 provides a more detailed illustration of the embodiment wherein the monitoring of tip temperatures is performed by the pacer/ICD, with the warning signals then forwarded to an external system. At step 200, the pacer/ICD monitors magnetic fields to detect the presence of strong fields indicative of an MRI or other magnetic imaging procedures. A magnetometer may be used to detect the MRI fields. So long as strong magnetic fields are not detected, routine device operations are performed at step 202, such as delivery of pacing pulses in a synchronous “demand” mode, such as VDD or DDD. VDD or DDD are standard device codes that identify the mode of operation of the device. Others standard modes include VVI, DDI and VOO. Briefly, DDD indicates a device that senses and paces in both the atria and the ventricles and is capable of both triggering and inhibiting functions based upon events sensed in the atria and the ventricles. VDD indicates a device that sensed in both chambers but only paces in the ventricle. A sensed event on the atrial channel triggers a ventricular output after a programmable delay. VVI indicates that the device is capable of pacing and sensing only in the ventricles and is only capable of inhibiting the functions based upon events sensed in the ventricles. DDI is identical to DDD except that the device is only capable of inhibiting functions based upon sensed events, rather than triggering functions. As such, the DDI mode is a non-tracking mode precluding its triggering ventricular outputs in response to sensed atrial events. VOO identifies fixed-rate ventricular pacing, which ignores any potentially sensed cardiac signals. This mode is quite different from the aforementioned “demand” modes, which only pace when the pacemaker determines that the heart is “demanding” pacing. Numerous other device modes of operation are possible, each represented by standard abbreviations of this type.

However, if a strong magnetic field is detected, then, at step 204, the pacer/ICD retrieves critical temperatures for each lead (that has a temperature sensor mounted near its tip) from memory. Alternatively, the pacer/ICD calculates critical temperatures based on stored models. As already explained, the critical temperature represents the temperatures at which there is a significant risk of: tissue damage; sensing impairment; and/or therapeutic stimulation impairment for a particular lead. Typically, different leads will have different critical temperatures. For a three lead implementation (e.g. LV, RV and RA leads) wherein each of the leads have temperature sensors near their respective tips, the pacer/ICD thereby retrieves or calculates separate critical tip temperatures for all three leads. These critical temperatures may be determined in advance based on otherwise routine experiments or studies. For example, studies may be performed to determine average critical temperatures for different combinations of leads and pacer/ICDs provided by various manufacturers. A particular pacer/ICD is then programmed following device implanted to store the appropriate critical temperatures for the particular leads that are implanted along with the device. Thereafter, the pacer/ICD merely looks up the critical temperature from memory for use in calculating the temperature threshold. In some implementations, the pacer/ICD may be programmed to adjust the critical temperature retrieved from memory based on current operating characteristics of the implantable system (such as the actual impedance of the lead) so as to more precisely estimate the critical temperature for the lead. Such adjustments may be made based on pre-programmed adjustment models. In some implementations, the pacer/ICD directly calculates or estimates the critical temperature for the lead based on stored models. Note also that multiple critical thresholds may be specified per lead. For example, one critical temperature may be set based on the temperature as which tissue damage is expected to occur. Another may be based on the temperature at which pacing efficacy might be significantly impaired. Yet another may be based on the temperature at which sensing efficacy might be significantly impaired. This allows multiple temperature thresholds to be defined per lead so that different warnings may be generated, depending upon the particular risk, or so that particular appropriate actions can be taken, such as increasing pulse magnitudes in response to the lead temperature exceeding a capture impairment threshold, changing sensitivity in response to the lead exceeding a sensing impairment threshold, etc.

At step 206, the pacer/ICD then calculates temperature thresholds for each lead by subtracting safety margins. The safety margins are preferably retrieved from memory as well. Different safety margins may be stored for use with different leads. If multiple critical temperatures are defined per leads, separate safety margins may be defined as well. As with the critical temperatures, the safety margins may be determined in advance based on otherwise routine experiments or studies, with different safety margins specified for different combinations of leads/devices. However, in some implementations, a single safety margin may be specified for use in calculating all thresholds for all leads. As noted above, a suitable safety margin may be, e.g., in the range of 3 to 4° C.

At step 208, the pacer/ICD then begins measuring tip temperatures using fiber optic-based temperature sensors installed within the leads (or other suitable temperature sensors). Fiber optic devices are preferred as such devices typically can provide reliable temperature readings even in the presence of MRI fields. One manufacturer of fiber optic temperature sensors is FISO Technologies of Quebec, Canada. Temperature sensors manufactured by FISO or other companies may be modified, if needed, to operate reliably within an implantable medical device lead. Preferably, temperature readings are received more or less continuously from the sensor so as to provide the pacer/ICD with time-varying temperature readings. However, in other implementations, such readings may be made periodically, such as once per second. Assuming that each lead has a temperature sensor, the pacer/ICD thereby receives a set of temperature signals, which are separately processed. Individual temperature values from each sensor may be stored in device memory for subsequent transmission to the external monitor or an external programmer. At step 210, the pacer/ICD compares tip temperatures to the aforementioned thresholds to detect when tip temperatures begin to approach critical temperatures. For example, a simple comparator may be used to compare a current tip temperature to the corresponding threshold. Depending upon the amount of noise or other fluctuations in the temperature signal, it may be appropriate to average some number of temperature readings before performing the comparison so as to avoid false positives based on anomalous temperature readings.

Then, at step 214, for any leads where tip temperatures exceed their corresponding thresholds, the pacer/ICD generates and transmits warning signals and temperature values to the external monitor and/or adjusts pacing functions by, e.g. increasing pulse magnitude, switching pacing mode to an asynchronous mode, etc. As noted, in addition to transmitting the actual warning signals, temperature values can be transmitted as well (either raw values or averaged values). Preferably the warnings also indicate the particular lead that exceeded its temperature threshold and, if multiple thresholds are provide per lead (i.e. a tissue damage threshold, a sensing impairment thresholds, etc.) the warnings can also specify the particular threshold that has been exceeded. Preferably, any transmissions to the external monitor exploit the improved transmission techniques set forth within the patent application to Min et al., discussed above, so as to help ensure reliable signal transmission and reception despite the strong magnetic fields. Warnings may also be directly provided to the patient via an implanted warning device, such as a vibrating or “tickle” voltage device, if provided.

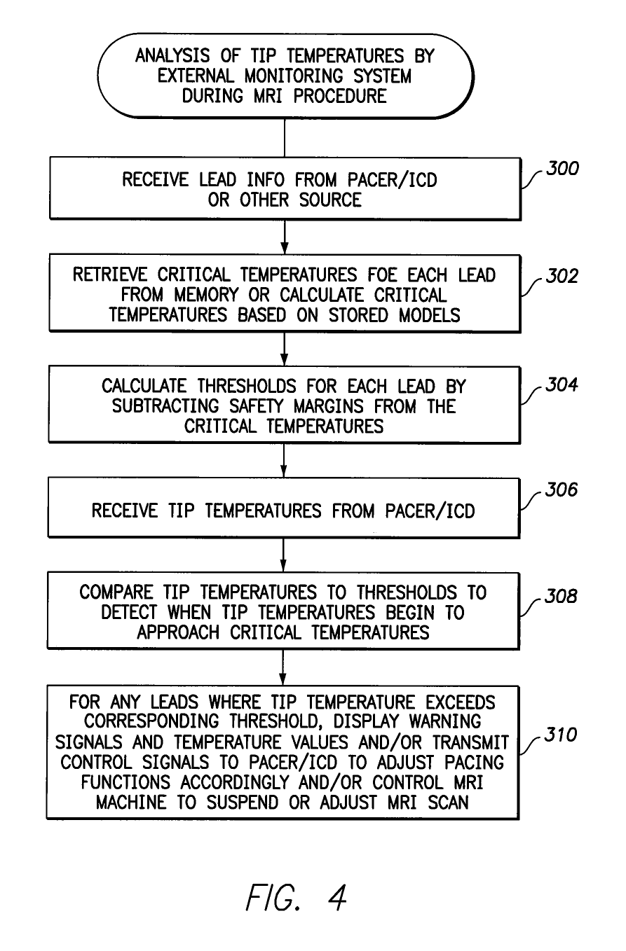

FIG. 4 provides a detailed illustration of an embodiment wherein the monitoring of tip temperatures is performed by the external monitor based on temperature signals received from the pacer/ICD. This embodiment may be particularly appropriate for use with pacer/ICDs that are equipped to sense and transmit temperature signals but are not otherwise equipped to compare temperatures to thresholds and to take corrective action. At step 300, the external monitor receives (via long range RF telemetry) information specifying the model of the device and its leads from which the external monitor can then read out from memory, at step 302, the appropriate critical temperatures for the particular combinations of leads/devices implanted within the patient. Alternatively, the external monitor receives the critical temperature from the pacer/ICD or calculates appropriate critical temperatures based on temperature models or the like. As with the embodiments already described, different leads may have different critical temperatures and multiple critical thresholds may be specified per lead. In any case, at step 304, the external monitor then calculates temperature thresholds for each-lead by subtracting suitable safety margins, which may be retrieved from memory as well. At step 306, the pacer/ICD then begins receiving tip temperature signals (e.g. raw temperature signals, running average temperatures, etc.) measured by the pacer/ICD and transmitted to the external system via long range telemetry. Assuming that the implanted system has multiple leads, each with its own temperature sensor, the external monitor thereby receives a set or plurality of temperature signals, which are separately processed by the external monitor. The temperature signals may be graphically displayed using the external monitor. At step 308, the external monitor compares tip temperatures to the aforementioned thresholds to detect when tip temperatures begin to approach critical temperatures. Again, depending upon the amount of noise or other fluctuations in the received temperature signals, it may be appropriate to average some number of temperature readings before performing the comparison so as to avoid false positives based on anomalous temperature readings.

Then, at step 310, for any leads where tip temperatures exceed corresponding thresholds, the external monitor generates and displays warning signals. Preferably, the displays also indicate the particular lead that exceeded its temperature threshold and, if multiple thresholds are provided per lead (i.e. a tissue damage threshold, a sensing impairment thresholds, etc.), the warnings also specify the particular threshold. Audible alarms may be generated as well. In some implementations, the external monitor may also automatically adjust pacing functions with the pacer/ICD to, e.g. increasing pulse magnitude, changes pacing modes, etc., by sending suitable long range telemetry signals to the implanted device. In still further implementations, the external monitor may also automatically control the operation of the MRI machine to, e.g., suspend its operation due to rising lead temperatures, or the like. As noted, the embodiment of FIG. 4 may be particularly appropriate for use with pacer/ICDs that are equipped to sense and transmit temperature signals but are not otherwise equipped to compare temperatures to thresholds and take corrective action. However, even when used in conjunction with such devices, the external monitor-based implementations may nevertheless be advantageous. For example, the external monitors may be programmed to exploit more sophisticated analysis techniques that the pacer/ICD is not capable of performing due to memory or processing limitations. For example, techniques that are more sophisticated may be exploited for estimating critical temperatures, determining when the lead temperatures approach the critical temperatures, etc.

Additionally, techniques set forth in the following patents and applications can be employed, where appropriate: U.S. patent application Ser. No. 11/955,268, filed Dec. 12, 2007, of Min, entitled “Systems and Methods for Determining Inductance and Capacitance Values for use with LC Filters within Implantable Medical Device Leads to Reduce Lead Heating during an MRI”; U.S. Pat. No. 6,395,637 to Park, et al. of the Electronics and Telecommunications Research Institute, entitled “Method for Fabricating an Inductor of Low Parasitic Resistance and Capacitance”; U.S. patent application Ser. No. 11/860,342, filed Sep. 27, 2007, entitled “Systems And Methods For Using Capacitive Elements To Reduce Heating Within Implantable Medical Device Leads During An MRI”; and U.S. patent application Ser. No. 12/042,605, filed Mar. 5, 2008, entitled “Systems And Methods For Using Resistive Elements And Switching Systems To Reduce Heating Within Implantable Medical Device Leads During An MRI.”

See, also: U.S. patent application Ser. No. 11/963,243, filed Dec. 21, 2007, entitled “MEMS-based RF Filtering Devices for Implantable Medical Device Leads to Reduce Lead Heating during MRI”; U.S. patent application Ser. No. 11/943,499, filed Nov. 20, 2007, entitled “RF Filter Packaging for Coaxial Implantable Medical Device Lead to Reduce Lead Heating during MRI”; U.S. patent application Ser. No. 12/117,069, filed May 8, 2008, entitled “Shaft-mounted RF Filtering Elements for Implantable Medical Device Lead to Reduce Lead Heating During MRI”; U.S. patent application Ser. No. 12/257,263, filed Oct. 23, 2008, entitled “Systems and Methods for Exploiting the Ring Conductor of a Coaxial Implantable Medical Device Lead to provide RF Shielding during an MRI to Reduce Lead Heating”; U.S. patent application Ser. No. 12/257,245, filed Oct. 23, 2008, entitled “Systems and Methods for Disconnecting Electrodes of Leads of Implantable Medical Devices during an MRI to Reduce Lead Heating while also providing RF Shielding ”.

See, further, U.S. patent application Ser. No. 12/270,768, filed Nov. 13, 2008, entitled “Systems And Methods For Reducing RF Power Or Adjusting Flip Angles During An MRI For Patients With Implantable Medical Devices”; U.S. patent application Ser. No. 12/325,945, filed Dec. 1, 2008, entitled “Systems And Methods For Selecting Components For Use In RF Filters Within Implantable Medical Device Leads Based On Inductance, Parasitic Capacitance And Parasitic Resistance”; U.S. patent application Ser. No. 11/256,480, filed Oct. 20, 2005, entitled “Improved Feedthrough Filter For Use In An Implantable Medical Device”; and U.S. patent application Ser. No. 11/020,438, filed Dec. 22, 2004, entitled “System and Method for Responding to Pulsed Gradient Magnetic Fields using an Implantable Medical Device.”

See, still further, U.S. patent application Ser. No. 12/537,880, filed Aug. 7, 2009, entitled “Implantable Medical Device Lead Incorporating Insulated Coils Formed as Inductive Bandstop Filters to Reduce Lead Heating During MRI” (Attorney Docket A09P1042); and U.S. patent application Ser. No. 12/537,916, filed Aug. 7, 2009, entitled “Implantable Medical Device Lead Incorporating a Conductive Sheath Surrounding Insulated Coils to Reduce Lead Heating During MRI” (Attorney Docket A09P1043.)

The techniques discussed above can be implemented in a wide variety of implantable medical devices for use with a wide variety of external systems. For the sake of completeness, detailed descriptions of an exemplary pacer/ICD and an exemplary external monitoring system will now be provided.

### Exemplary Pacer/ICD

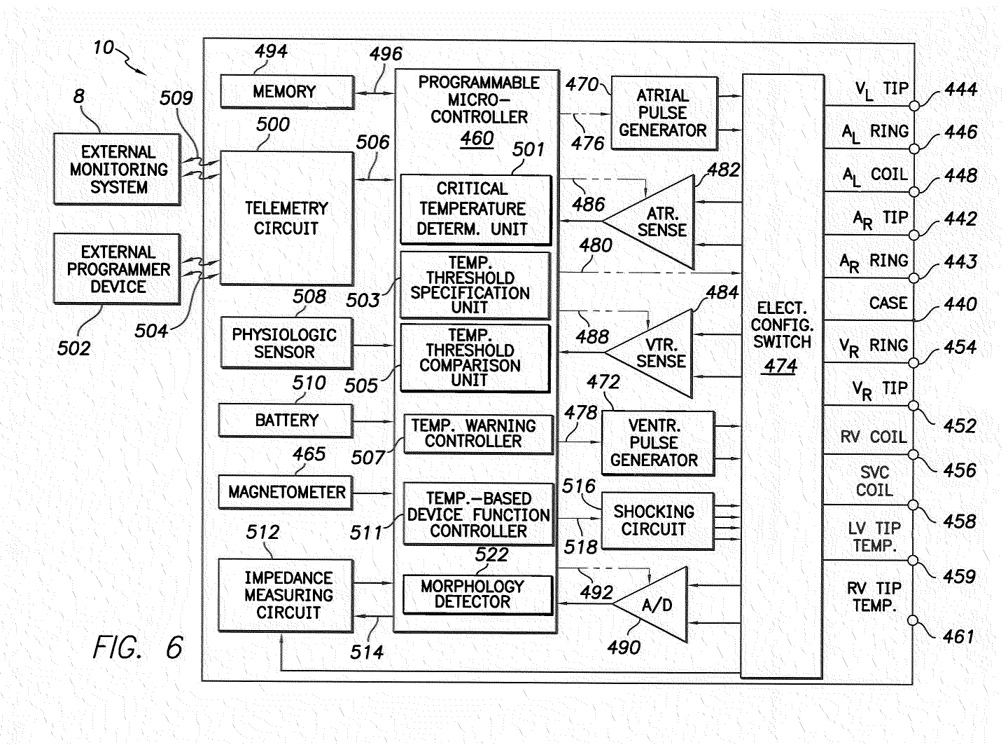

With reference to FIGS. 5 and 6, a description of the pacer/ICD of FIG. 1 will now be provided where the pacer/ICD is equipped to analyze lead temperatures and control device functions in response thereto (as already described above with reference to FIG. 3). FIG. 5 provides a simplified diagram of the pacer/ICD, which is a dual-chamber stimulation device capable of treating both fast and slow arrhythmias with stimulation therapy, including cardioversion, defibrillation, and pacing stimulation, as well as capable of detecting and tracking tip temperatures. To provide atrial chamber pacing stimulation and sensing, pacer/ICD 10 is shown in electrical communication with a heart 412 by way of a left atrial lead 420 having an atrial tip electrode 422 and an atrial ring electrode 423 implanted in the atrial appendage. Pacer/ICD 10 is also in electrical communication with the heart by way of a right ventricular lead 430 having, in this embodiment, a ventricular tip electrode 432, a right ventricular ring electrode 434, a right ventricular (RV) coil electrode 436, and a superior vena cava (SVC) coil electrode 438. Typically, the right ventricular lead 430 is transvenously inserted into the heart so as to place the RV coil electrode 436 in the right ventricular apex, and the SVC coil electrode 438 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a “coronary sinus” lead 424 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 424 is designed to receive atrial and ventricular cardiac signals and to deliver left ventricular pacing therapy using at least a left ventricular tip electrode 426, left atrial pacing therapy using at least a left atrial ring electrode 427, and shocking therapy using at least a left atrial coil electrode 428. With this configuration, biventricular pacing can be performed. Although only three leads are shown in FIG. 5, it should also be understood that additional stimulation leads (with one or more pacing, sensing and/or shocking electrodes) might be used in order to efficiently and effectively provide pacing stimulation to the left side of the heart or atrial cardioversion and/or defibrillation.

Additionally, a fiber optic temperature sensor 437 is shown mounted near the tip of CS lead 424 that transmits temperature signals to the pacer/ICD. Another fiber optic temperature sensor 439 is shown mounted near the tip of RV lead 430 that also transmits temperature signals to the pacer/ICD. Although not shown, a temperature sensor may also be provided on the RA lead. Also, multiple temperature sensors may be provided per lead so as to track temperatures are different locations along the lead. Numerous other sensors can be mounted to the various pacing/sensing leads or to other leads as well, such pressure sensors, blood oxygen saturation sensors, etc.

A simplified block diagram of internal components of pacer/ICD 10 is shown in FIG. 6. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned apnea detection and therapy.

The housing 440 for pacer/ICD 10, shown schematically in FIG. 6, is often referred to as the “can”, “case” or “case electrode” and may be programmably selected to act as the return electrode for all “unipolar” modes. The housing 440 may further be used as a return electrode alone or in combination with one or more of the coil electrodes, 428, 436 and 438, for shocking purposes. The housing 440 further includes a connector (not shown) having a plurality of terminals, 442, 443, 444, 446, 448, 452, 454, 456 and 458 (shown schematically and, for convenience, the names of the electrodes to which they are connected are shown next to the terminals). As such, to achieve right atrial sensing and pacing, the connector includes at least a right atrial tip terminal (A_{R} TIP) 442 adapted for connection to the atrial tip electrode 422 and a right atrial ring (A_{R} RING) electrode 443 adapted for connection to right atrial ring electrode 423. To achieve left chamber sensing, pacing and shocking, the connector includes at least a left ventricular tip terminal (V_{L} TIP) 444, a left atrial ring terminal (A_{L} RING) 446, and a left atrial shocking terminal (A_{L} COIL) 448, which are adapted for connection to the left ventricular ring electrode 426, the left atrial tip electrode 427, and the left atrial coil electrode 428, respectively. To support right chamber sensing, pacing and shocking, the connector further includes a right ventricular tip terminal (V_{R} TIP) 452, a right ventricular ring terminal (V_{R} RING) 454, a right ventricular shocking terminal (R_{V} COIL) 456, and an SVC shocking terminal (SVC COIL) 458, which are adapted for connection to the right ventricular tip electrode 432, right ventricular ring electrode 434, the RV coil electrode 436, and the SVC coil electrode 438, respectively. An LV temperature sensor terminal 459 is provided for connection to LV/CS temperature sensor 437. An RV temperature sensor terminal 461 is provided for connection to LV temperature sensor 437.

At the core of pacer/ICD 10 is a programmable microcontroller 460, which controls the various modes of stimulation therapy. As is well known in the art, the microcontroller 460 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing timing and data analysis functions are well known in the art.

As shown in FIG. 6, an atrial pulse generator 470 and a ventricular pulse generator 472 generate pacing stimulation pulses for delivery by the right atrial lead 420, the right ventricular lead 430, and/or the coronary sinus lead 424 via an electrode configuration switch 474. It is understood that in order to provide stimulation therapy in each of the four chambers of the heart, the atrial and ventricular pulse generators, 470 and 472, may include dedicated, independent pulse generators, multiplexed pulse generators or shared pulse generators. The pulse generators, 470 and 472, are controlled by the microcontroller 460 via appropriate control signals, 476 and 478, respectively, to trigger or inhibit the stimulation pulses.

The microcontroller 460 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, combipolar, etc.) by selectively closing the appropriate combination of switches (not shown) as is known in the art.

Atrial sensing circuits 482 and ventricular sensing circuits 484 may also be selectively coupled to the right atrial lead 420, coronary sinus lead 424, and the right ventricular lead 430, through the switch 474 for detecting the presence of cardiac activity in each of the four chambers of the heart. Accordingly, the atrial (ATR. SENSE) and ventricular (VTR. SENSE) sensing circuits, 482 and 484, may include dedicated sense amplifiers, multiplexed amplifiers or shared amplifiers. The switch 474 determines the “sensing polarity” of the cardiac signal by selectively closing the appropriate switches, as is also known in the art. In this way, the clinician may program the sensing polarity independent of the stimulation polarity. Each sensing circuit, 482 and 484, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 482 and 484, are connected to the microcontroller 460 which, in turn, are able to trigger or inhibit the atrial and ventricular pulse generators, 470 and 472, respectively, in a demand fashion in response to the absence or presence of cardiac activity in the appropriate chambers of the heart.

For arrhythmia detection, pacer/ICD 10 utilizes the atrial and ventricular sensing circuits, 482 and 484, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 460 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 490. The data acquisition system 490 is configured to acquire intracardiac electrogram signals, convert the raw analog data into a digital signal, and store the digital signals for later processing and/or telemetric transmission to an external device 502. The data acquisition system 490 is coupled to the right atrial lead 420, the coronary sinus lead 424, and the right ventricular lead 430 through the switch 474 to sample cardiac signals across any pair of desired electrodes. The microcontroller 460 is further coupled to a memory 494 by a suitable data/address bus 496, wherein the programmable operating parameters used by the microcontroller 460 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 494 through a telemetry circuit 500 in telemetric communication with an external device 502, such as a programmer, transtelephonic transceiver or a diagnostic system analyzer, or the external monitoring system 8 (FIG. 1). The telemetry circuit 500 is activated by the microcontroller by a control signal 506. The telemetry circuit 500 advantageously allows IEGMs and other electrophysiological signals and/or hemodynamic signals, including tip temperature information signals, to be sent to the external programmer device 502 through an established communication link 504 or to a separate external monitoring system via link 509 (or link 9 of FIG. 1).

Pacer/ICD 10 further includes an accelerometer or other physiologic sensor 508, commonly referred to as a “rate-responsive” sensor because it is typically used to adjust pacing stimulation rate according to the exercise state of the patient. However, the physiological sensor 508 may further be used to detect changes in cardiac output, changes in the physiological condition of the heart, or diurnal changes in activity (e.g., detecting sleep and wake states) and to detect arousal from sleep. Accordingly, the microcontroller 460 responds by adjusting the various pacing parameters (such as rate, AV Delay, V-V Delay, etc.) at which the atrial and ventricular pulse generators, 470 and 472, generate stimulation pulses. While shown as being included within pacer/ICD 10, it is to be understood that the physiologic sensor 508 may also be external to pacer/ICD 10, yet still be implanted within or carried by the patient, such as sensor 437 of FIG. 6. A common type of rate responsive sensor is an activity sensor incorporating an accelerometer or a piezoelectric crystal, which is mounted within the housing 440 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 510, which provides operating power to all of the circuits shown in FIG. 6. The battery 510 may vary depending on the capabilities of pacer/ICD 10. If the system only provides low voltage therapy, a lithium iodine or lithium copper fluoride cell may be utilized. For pacer/ICD 10, which employs shocking therapy, the battery 510 must be capable of operating at low current drains for long periods, and then be capable of providing high-current pulses (for capacitor charging) when the patient requires a shock pulse. The battery 510 must also have a predictable discharge characteristic so that elective replacement time can be detected. Accordingly, pacer/ICD 10 is preferably capable of high voltage therapy and appropriate batteries.

As further shown in FIG. 6, pacer/ICD 10 is shown as having an impedance measuring circuit 512 which is enabled by the microcontroller 460 via a control signal 514. Various uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 120 is advantageously coupled to the switch 74 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 460 further controls a shocking circuit 516 by way of a control signal 518. The shocking circuit 516 generates shocking pulses of low (up to 0.5 joules), moderate (0.5-10 joules) or high energy (11 to 40 joules), as controlled by the microcontroller 460. Such shocking pulses are applied to the heart of the patient through at least two shocking electrodes, and as shown in this embodiment, selected from the left atrial coil electrode 428, the RV coil electrode 436, and/or the SVC coil electrode 438. The housing 440 may act as an active electrode in combination with the RV electrode 436, or as part of a split electrical vector using the SVC coil electrode 438 or the left atrial coil electrode 428 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 460 is capable of controlling the synchronous or asynchronous delivery of the shocking pulses.

Insofar as lead temperature monitoring operations are concerned, the microcontroller includes a magnetometer 465 for detecting the presence of MRI fields or other strong magnetic fields so as to enable lead temperature monitoring. A critical temperature determination unit 501 determines the critical temperature for each lead to be monitored using techniques discussed above (including retrieval of prestored values from memory 494.) A temperature threshold specification unit 503 calculates or “specifies” a threshold for each critical temperature by, e.g., subtracting safety margin values retrieved from memory. A temperature threshold comparison unit 505 compared temperature values received via terminals 459 and 461 with the thresholds. A temperature warning controller 507 generates warnings or other diagnostic information in response to lead temperatures exceeding their respective thresholds. The warnings may be transmitted to the external monitoring system 8 via telemetry circuit 500. A temperature-based device function controller 511 controls device functions in response to lead temperatures exceeding their respective thresholds to, e.g., switch pacing modes, changing pacing pulse magnitudes, etc.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

### Exemplary External Monitoring System

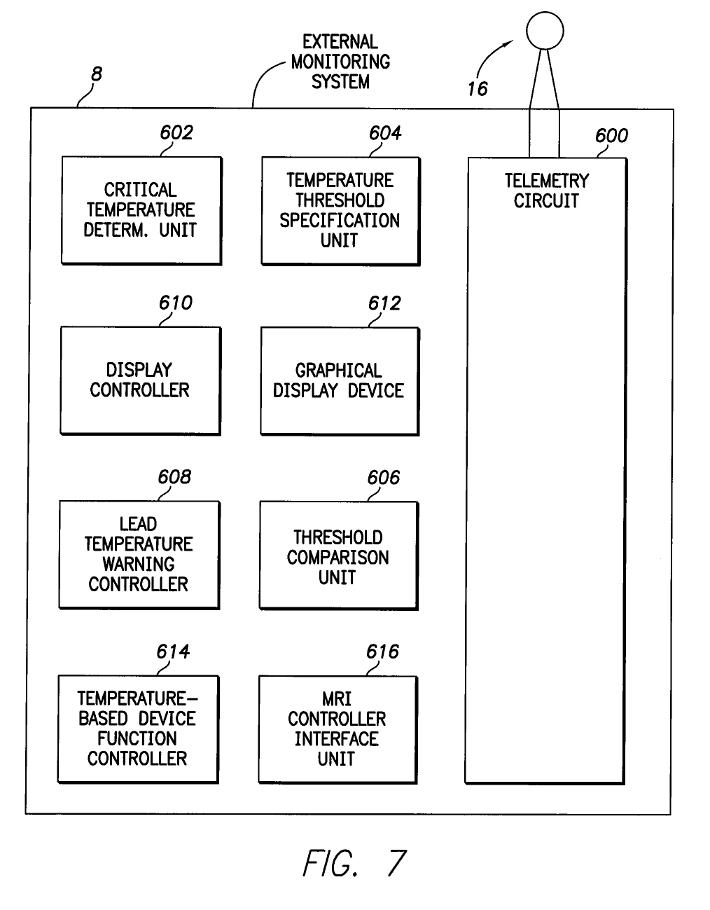

With reference to FIG. 7, a brief description of an exemplary remote monitoring system 8 for use in the system of FIG. 1 will now be provided, where the remote monitoring system is equipped to analyze lead temperatures and control various functions in response thereto (as already described above with reference to FIG. 4). Remote monitoring system 8 includes a telemetry circuit 600 connected to long range RF antenna 16 for communicating with a pacer/ICD or other implantable medical device and, in particular, for receiving tip temperature signals transmitted by the pacer/ICD. A critical temperature determination unit 602 determines the critical temperature for each lead of the implanted device (that is at risk of overheating during an MRI), assuming that information is not also transmitted from the pacer/ICD. A temperature threshold specification unit 604 calculates or specifies a threshold for each critical temperature by, e.g., subtracting predetermined safety margin values. A temperature threshold comparison unit 606 compares temperature values received from the pacer/ICD with the thresholds. A temperature warning controller 608 generates warnings or other diagnostic information in response to lead temperature exceeding their respective thresholds. A display controller 610 controls the generation of graphical displays of warnings, as well as displays of temperature data received from the pacer/ICD, for display on a graphical display device 612, such as an LCD, CRT display or the like. Warnings may also be provided via an audio transducer. A temperature-based device function controller 614 generates reprogramming signals for controlling pacer/ICD functions in response to lead temperatures exceeding their respective thresholds to, e.g., switch pacing modes, changing pacing pulse magnitudes, etc. Such signals may be transmitted via antenna 16 to the pacer/ICD using transmission techniques set forth in the application of Min et al. cited above. An MRI controller interface unit 616 is provided for interfacing with the controller (block 6 of FIG. 1) of the MRI machine for sending signals to the MRI controller to suspend the MRI procedure, if warranted due to the detection of excessive lead temperatures.

### Temperature Sensor Locations

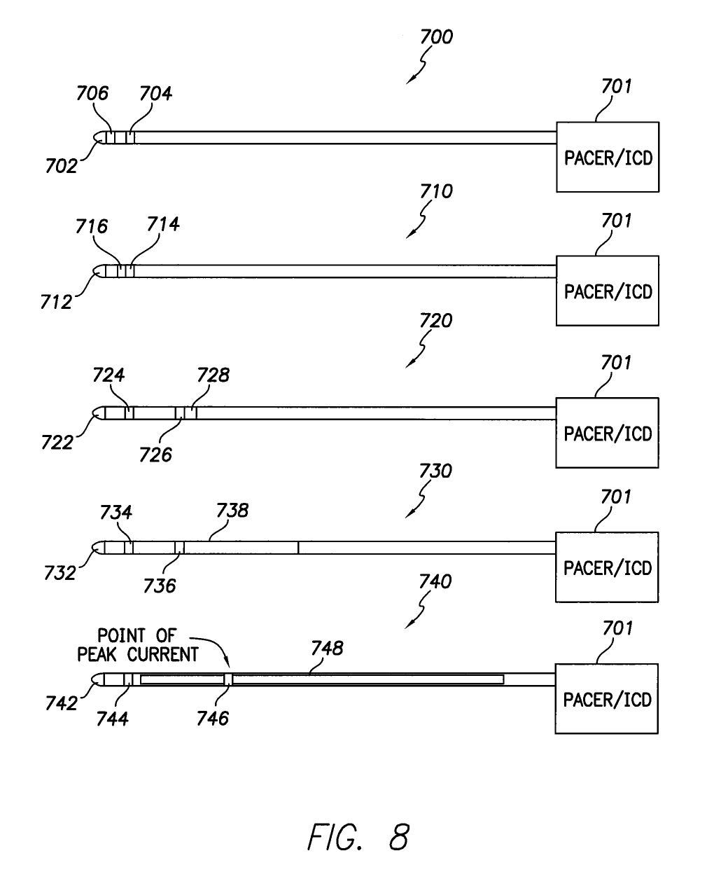

Although primarily described with respect to implementations wherein the temperature sensor is near the tip of the lead, other locations may be appropriate as well, depending up on the particular lead. Various examples are provided in FIG. 8 by way of simplified lead illustrations.

A first exemplary lead 700 of FIG. 8 (coupled to a pacer/ICD 701) includes a tip electrode 702 and a ring electrode 704. A temperature sensor 706 is mounted on or near the tip electrode. This is similar to the arrangement of FIG. 1. A second exemplary lead 710 again includes a tip electrode 712 and a ring electrode 714. A temperature sensor 716 is mounted on or near the ring electrode in this example. A third exemplary lead 720 again includes a tip electrode 722 and a ring electrode 724. The lead also includes an RF filter 728 in the form of a LC resonator or a self-resonant inductor. A temperature sensor 726 is mounted on or near the RF filter. (See the various patent documents cited above for descriptions of RF filters for use in implantable medical device leads.) A fourth exemplary lead 730 again includes a tip electrode 732 and a ring electrode 734. The lead also includes a shocking coil 738. A temperature sensor 736 is mounted on or near the shocking coil. A fifth exemplary lead 740 again includes a tip electrode 742 and a ring electrode 744. The lead includes a distributed RF filtering component 748, which can be formed of insulated wires inside the lead. A temperature sensor 746 is mounted on or near a point of peak current along the lead. (See the various patent documents cited above for descriptions of distributed RF filters for use in implantable medical device leads.) These are just some specific examples. As can be appreciated, temperature sensors can be located at still other positions along the lead. Two or more temperature sensors can be provided as well.

What have been described are various systems and methods for use with a pacer/ICD in conjunction with an external monitoring system. Principles of the invention may be exploiting using other implantable systems, externals systems or in accordance with other techniques. Thus, while the invention has been described with reference to particular exemplary embodiments, modifications can be made thereto without departing from the scope of the invention.

What is claimed is:

1. A method for use by an implantable medical device for implant within a patient wherein the device employs at least one electrical lead implanted within patient tissue, the method comprising:
determining a critical temperature for the lead;
setting a temperature threshold based on the critical temperature to a value below the critical temperature;
sensing at least one lead temperature value during a magnetic imaging procedure;
comparing the lead temperature value against the threshold; and
controlling at least one device function based on whether the lead temperature value exceeds the threshold such that device functions can be controlled in response to lead temperatures approaching the critical temperature.

2. The method of claim 1 wherein the critical temperature for the lead is representative of a temperature at which there is a significant risk of one or more of: tissue damage in tissues adjacent the lead; impairment to effective signal sensing; and impairment to effective therapeutic stimulation.

3. The method of claim 1 wherein determining the critical temperature of the lead includes retrieving a pre-stored critical temperature from device memory.

4. The method of claim 1 wherein determining the critical temperature of the lead includes calculating the critical temperature based on temperature models stored within the implantable device.

5. The method of claim 1 wherein a plurality of leads is provided for use with the implantable medical device and wherein determining the critical temperature of the lead includes determining a separate critical temperature for each lead.

6. The method of claim 1 wherein setting the temperature threshold based on the critical temperature is performed by subtracting a predetermined safety margin from the critical temperature.

7. The method of claim 6 wherein the safety margin is in the range of 3 to 4 degrees Celsius (C.).

8. The method of claim 6 wherein a plurality of leads are provided for use with the implantable medical device and wherein a separate safety margin is used for each lead.

9. The method of claim 1 wherein sensing at least one lead temperature value includes sensing at least one temperature value at one or more of a tip location, a ring location, a shocking coil location, an RF filter location and at a location of substantially peak current along the lead.

10. The method of claim 9 wherein sensing at least one tip temperature value includes sensing changes in tip temperature over time.

11. The method of claim 1 wherein a plurality of leads are provided and wherein sensing at least one lead temperature value during a magnetic imaging procedure includes separately sensing at least one tip temperature value for each of the leads.

12. The method of claim 1 wherein sensing the lead temperature value during a magnetic imaging procedure includes detecting the presence of a magnetic field indicative of a magnetic imaging procedure and sensing the lead temperature value in response thereto.

13. The method of claim 12 wherein detecting the presence of a magnetic field includes detecting the presence of a field associated with a magnetic resonance imaging (MRI) procedure.

14. The method of claim 1 wherein controlling at least one device function based on whether the lead temperature value exceeds the threshold includes transmitting the lead temperature value to an external monitoring system if the lead temperature value exceeds the threshold such that lead temperatures approaching the critical temperature can be remotely monitored during the magnetic imaging procedure.

15. The method of claim 1 wherein controlling at least one device function based on whether the lead temperature value exceeds the threshold includes transmitting a warning signal to an external monitoring system if the lead temperature value exceeds the threshold such that a warning can be issued if lead temperatures approach the critical temperature during the magnetic imaging procedure.

16. The method of claim 15 wherein a plurality of temperature thresholds are employed for use with a given lead and wherein transmitting a warning signal to the external monitoring system includes transmitting different warning signals based on the particular threshold exceeded.

17. A system for use in an implantable medical device for implant within a patient wherein the device employs at least one electrical lead implanted within patient tissue, the system comprising:
a critical temperature determination unit operative to determine a critical temperature for the lead;
a temperature threshold specification unit operative to set a temperature threshold based on the critical temperature to a value below the critical temperature;
a temperature input system operative to input at least one lead temperature value sensed during a magnetic imaging procedure;
a threshold comparison unit operative to compare the lead temperature value against the threshold; and
a device function controller operative to control at least one device function based on whether the lead temperature value exceeds the threshold such that device functions can be controlled in response to lead temperatures approaching the critical temperature.

18. A method for use by an implantable medical device for implant within a patient wherein the device employs at least one electrical lead implanted within patient tissue, the method comprising:
determining a critical temperature for the lead;
setting a temperature threshold based on the critical temperature to a value below the critical temperature;
sensing at least one lead temperature value during a magnetic imaging procedure;
comparing the lead temperature value against the threshold; and
transmitting the lead temperature value to an external monitoring system if the lead temperature value exceeds the threshold such that lead temperatures approaching the critical temperature can be remotely monitored during the magnetic imaging procedure.
